(19) United States
(12) Patent Application Publication
(10) Pub. No.: US 2010/0145025 A1
(43) Pub. Date: Jun. 10, 2010

(54) Recombinant Mouse-Human Chimeric Fab Against Hepatitis B Surface Antigen

(75) Inventors: Biplab Bose, Assam (IN); Subrata Sinha, New Delhi (IN); Navin Khanna, New Delhi (IN); Subrat Kumar Acharya, New Delhi (IN)

Correspondence Address: THE WEBB LAW FIRM, P.C., 700 KOPPERS BUILDING, 436 SEVENTH AVENUE, PITTSBURGH, PA, 15219, US

(73) Assignees: DEPARTMENT OF BIOTECHNOLOGY (DBT), New Delhi (IN); ALL INDIA INSTITUTE OF MEDICAL SCIENCES (AIIMS), New Delhi (IN)

(21) Appl. No.: 12/519,898

(22) PCT Filed: Sep. 10, 2007
(86) PCT No.: PCT/IN2007/000403

§ 371 Date: Jan. 15, 2010

(30) Foreign Application Priority Data
Dec. 18, 2006 (IN) 2704/DEL/06

(51) Int. Cl. C07K 16/10

(52) U.S. Cl. 530/387.3

(57) ABSTRACT

The invention relates to a recombinant chimeric Fab antibody comprising a recombinant Fd and a recombinant chimeric light chain which binds to hepatitis B surface antigen with high affinity, generated by fusing variable region genes (V_{H} and V_{L}) of an anti-HBsAg mouse antibody 5S and constant region genes of human, CH1 region of human IgGI and the CL region of human kappa chain, wherein mouse V_{L} and human CL are linked by overlap PCR to generate chimeric light chain and mouse V_{H} and CH₁ are linked by overlap PCR to generate chimeric Fd.

### FIELD OF INVENTION

This invention relates to the generation of new recombinant mouse-human chimeric Fab antibody, which binds to the hepatitis B surface antigen with high affinity.

### PRIOR ART

Protective antibodies that appear after natural infection are mostly directed against the major antigenic ‘a’ determinant of Hepatitis B surface antigen (HBsAg). The immunodominant ‘a’ epitope is a part of a large antigenic area of HBsAg, called the major hydrophilic region and this epitope is present in all serotypes. Antibodies against HBsAg are thus advocated for passive immunotherapy against Hepatitis B infection in cases of accidental needle stick injuries, for liver transplant patients etc. Presently, human anti-HBs immune globulin (HB1G) collected from the blood of hyper immune donors is used for post exposure prophylaxis. This blood derived product is not manufactured in India. Being a blood derived product, anti-HBs HB1G is costly and can cause cross contamination.

Few anti-HBsAg mouse monoclonal have been reported in literature. However such mouse antibodies are not suitable for therapeutic uses as they may generate immunogenic reaction. Several recombinant anti-HBsAg antibody fragments have also been reported in literature. However, most of them are of mouse origin and are not available for therapeutic uses.

It is well documented that immunogenic reactions are predominantly directed towards the Fe region of murine antibodies. It has also been observed that creation of mouse-human chimeric antibodies, by swapping murnie constant regions with the human ones, can reduce the immunogenicity of an antibody. Such mouse-human chimeric molecules are safer for therapeutic uses and currently, several such chimeric molecules are in clinical uses for different diseases.

Five different recombinant anti-HBsAg Fab antibodies have been reported in literature. They are all unique in nature and are different from the recombinant molecule of this invention.

### OBJECTS OF THE INVENTION

The object of the invention is to generate a recombinant mouse-human chimeric antibody fragment, against Hepatitis B surface antigen.

Other objective is to develop a recombinant mouse-human chimeric Fab antibody which can bind to Hepatitis B surface antigen with high affinity and specificity.

Further objective is to show that the original monoclonal from which it is derived has binding characteristics that are very conducive to bio neutralization.

Another objective is to produce a recombinant mouse-human chimeric Fab antibody which would have less antigenicity as compared to the mouse monoclonal.

Yet another objective is to produce a recombinant mouse-human chimeric Fab antibody which would be more suitable for invivo use in humans.

Other objective is to generate mouse-human chimeric antibody which can be safer and cheaper alternative.

Further objective is to develop the recombinant molecule which can also be used with/without any modification/in combination with other molecules for generation of complete full length antibody bispecific antibodies, diabodies or any other modification of a protein molecule with containing the described Fab.

Yet another objective is also to develop immuno conjugates, like conjugated with enzymes, ligands, receptors, drugs, radio isotopes, toxins or any other large or small molecule for invivo or invitro use.

### DETAILED DESCRIPTION OF THE INVENTION

At the outset of the description that follows, it is to be understood that the ensuing description only illustrates a particular form of this invention. However, such a particular form is only an exemplary embodiment and is not intended to be taken restrictively to imply any limitation on the scope of the present invention.

There are many well known methods to generate recombinant chimeric antibodies. However, every recombinant chimeric antibody is unique in nature, if and only if (a) its molecular structure as defined by the amino acid sequence is unique (b) has unique biological function as defined by specificity and affinity for the target antigen. The recombinant molecule of this invention is unique as no other molecule matches the structure and properties of this molecule. This novel molecules can be generated by many other well documented strategies too.

Other recombinant molecule may have the same function as they bind the hepatitis B surface antigen but each molecule with different sequences has unique binding characteristics in terms of epitope specificity ie the precise sequence of antigen it binds to and the molecular interactions for doing the same and affinity.

Here, the inventors generated a recombinant chimeric Fab antibody, composed of a recombinant Fd and a recombinant chimeric light chain, which binds to Hepatitis B surface antigen with high affinity. Only the Fab and not the individual light and heavy chains can bind to Hepatitis B surface antigen and thus be useful for virus neutralization.

Inventors have used the antibody genes from a mouse monoclonal (named as 5S), for generation of recombinant antibody fragments against Hepatitis B surface antigen. This mouse antibody binds to the immunodominant ‘a’ epitope of the hepatitis B surface antigen and found to be protective in a surrogate in vitro assay. This mouse monoclonal was generated using, existing protocol for generation of hybridomas. However this mouse monoclonal cannot be used directly in human subjects, as it will trigger human anti-mouse immunogenic response. To reduce the immunogenicity of the antibody, inventors have generated a mouse-human chimeric Fab that retains the high affinity binding to HBsAg. Variable region genes (V_{H} and V_{L}) of the anti-HBs mouse monoclonal 5S were amplified by reverse transcription (RT) followed by polymerase chain reaction (PCR). Similarly constant region of human kappa chain (C_{K}) and first constant region of human IgGI heavy chain (C_{H}1) were amplified using human peripheral blood lymphocytes (PBLs) as the source of antibody genes. Mouse V_{L} and human C_{K} were linked by overlap PCR to generate chimeric Fd. Both the chimeric antibody genes were then cloned into a bicistronic bacterial expression vector (pCOMB3H, Scripps Research Institute, La Zola, USA) to express the chimeric Fab in the periplasm of E. coli (XL1-blue). The chimeric Fab so expressed in bacterial periplasm showed high binding to HBsAg and competitive ELISA confirmed that it binds to the same epitope as that of the original mouse monoclonal. The apparent dissociation constant of the chimeric antibody was found to be close to that of the original mouse monoclonal (4.575 nM). This chimeric anti-HBs Fab have been cloned and expressed in such a fashion that it can be further manipulated to generate a complete chimeric antibody by fusing the chimeric Fd with human Fc fragment. Therefore this chimeric Fab, which has unique mouse variable regions fused to human constant domains can be the starting material for generation of a therapeutically functional full-length recombinant antibody against Hepatitis B surface antigen. Being chimeric in nature, it is expected to be less immunogenic than a mouse antibody and as generated by recombinant means it can be safer and cheaper that the currently used human polyclonal antibody.

Inventive Steps:
1. Amplification of the variable region genes (V_{H} and V_{L}) of the anti-HBs mouse monoclonal 5S by reverse transcription (RT) followed by polymerase chain reaction (PCR). RNA isolated from 5S hybridoma was used as the source of these two genes.
2. Amplification of constant region of human kappa chain (Cₖ) and first constant region of human IgG1 heavy chain (CH1) by reverse transcription (RT) followed by polymerase chain reaction (PCR). RNA isolated from human peripheral blood lymphocytes (PBLs) was used as the source of these two genes.
3. Mouse V_{L} and human C_{K} were linked by overlap PCR to generate chimeric light chain. Similarly mouse V_{H} and CH1 were linked by overlap PCR to generate chimeric Fd.
4. Both of these chimeric antibody genes were cloned into a bicistronic bacterial expression vector (pCOMB3H, Scripps Research Institute, La Zola, USA) to express the chimeric Fab in the periplasm of E. coli (XL1-blue).
5. Recombinant clone was cultured in suitable medium and cell extract was prepared by the conventional method.
6. The recombinant chimeric antibody was purified by affinity chromatography using Protein G.

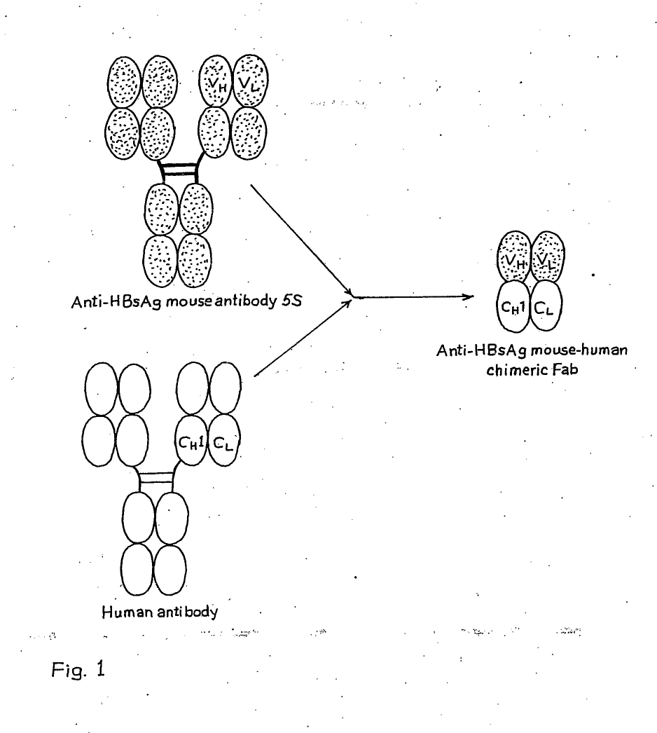

This recombinant molecule was generated by fusing variable region genes (V_{H} and V_{L}) of an anti-HBsAg mouse antibody 5S and constant region genes of human (C_{H}1 region of human IgG1 and the C_{L} region of human kappa chain). The over all scheme of the invention is shown in FIG. 1.

Cloning and Generation of the Recombinant Construct:

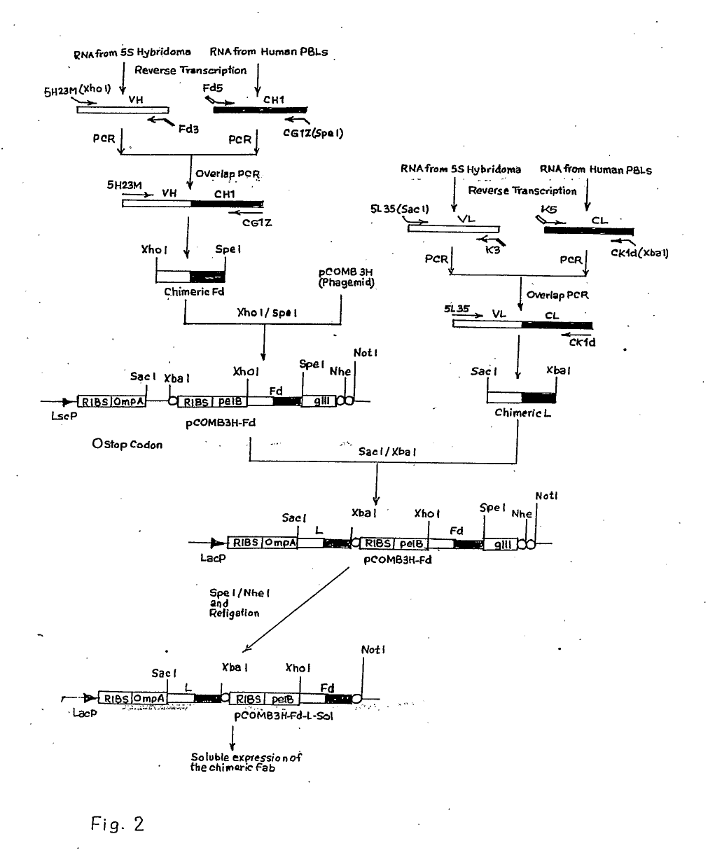

The strategy for generation of the chimeric Fab is shown in FIG. 2. The variable region genes of 5S hybridoma were amplified by reverse transcription followed by PCR. Primers used for all reverse transcriptions and PCRs are listed in Table 1. Total RNA was isolated from 5S cells using standard protocol and cDNAs for the V_{H} and V_{L} fragments were generated by reverse transcription. Primers used for reverse transcriptions were Fd3 and K3 for V_{H} and V_{L} respectively. The V_{H} fragment was further amplified by PCR using primers 5H23M and Fd3. Similarly the V_{L} fragment was amplified by PCR using primers 5L35 and K3. Conditions for both these PCRs were: 30 cycles at 94° C. for 1 min, 60° C. for 1 min, 72° C. for 2 min, followed by a final extension for 10 min at 72° C. For amplification of human constant domains, RNA was isolated from human peripheral blood lymphocytes (PBLs). DNAs for the C_{H}1 region of human IgG1 and the C_{L} region of human kappa chain were generated by reverse transcription. Primers used for reverse transcriptions were CG1Z and Ck1d for C_{H}1 and C_{L} respectively. The C_{H}1 fragment was further amplified by PCR using primers Fd5 and CG1Z. Human C_{L} was amplified by PCR using primers K5 and CK1d. Fd5 and K5 carry overhangs complementary to the 3′ end of variable regions of 5S. conditions for both these PCRs were: 30 cycles at 94° C. for 1 min, 60° C. for 1 min, 72° C. for 2 min, followed by a final extension for 10 min at 72° C.

PCR amplified fragments were resolved in 1.5% agarose gel and respective bands were eluted out using standard protocol. PCR amplified C_{H}1 (human IgG1) and mouse V_{H} were used as templates for generation of the chimeric Fd by overlap PCR. Intitial assembly of equimolar amounts of the mouse V_{H} and human C_{H}1 was done by PCR for 20 cycles at 94° C. for 1 min, 60° C. for 1 min and 72° C. for 2 min, followed by final extension of 10 min at 72° C. The product of the initial assembly reaction was diluted 10 times and used as the template for pull through PCR using primers 5H23M and CGIZ. Similarly human kappa C_{L} and mouse V_{L} fragment were joined to generate the chimeric light chain. Primers used for this reaction were 5L35 and CK1d.

The chimeric Fd was digested with Xhol and Spe1 and cloned into the phagemid vector pCOMB3H. The resulting construct (pCOMB3H-Fd) was transformed into chemically competent E. coli XL1-Blue cells by standard chemical method (Cacl₂/heat shock). Transformed cells were grown on Ampicillin-Agar plates. Colonies were picked up after overnight incubation and screened for the presence of the insert by colony PCR and restriction digestion (Xhol/spe1). The recombinant phagemid pCOMB3H-Fd was isolated by alkali-lysis method and digested with Sacl and Xbal. The chimeric light chain digested with these two enzymes was cloned into pCOMB3H-Fd to generate the phagemid construct pCOMB3H-Fd-L. This construct was transformed into chemically competent XL1-Blue cells by standard chemical transformation method and after overnight incubation, recombinant clones were checked for the presence of the chimeric light chain by colony PCR and by restriction digestion (Sacl/Xbal).

Expression of the Recombinant Chimeric Fab:

For soluble expression of the chimeric Fab, the phage gHI sequence was removed from the recombinant phagemid construct pCOMB3H-Fd-L by double digestion with spe I and Nhe I. Double digested phagemid was self-ligated to generate the construct pCOMB3H-Fd-L-Sol and transformed in chemically competent XL1-Blue cells. For soluble expression of recombinant chimeric Fab, 1 L Super Broth with 20 mmol/L MgCI₂ and Ampicillin (100 μg/ml) was inoculated with 10 ml of overnight culture of the recombinant clone and grown at 37° C. till the A₆₀₀ reached approximately 0.6, when over-expression of the chimeric Fab was induced by 1 mmol/L IPTG. After overnight growth at 30° C., cells were harvested by centrifugation at 4000 g. Cell pellet was re-suspended in 20 ml PBS/1mmol/L EDTA and kept on ice for 40 min. Clarified periplasmic extract was obtained by centrifugation of the re-suspended product at 10000 g.

Purification of the Recombinant Chimeric Fab:

The recombinant chimeric Fab was purified from the periplasmic extract by affinity chromatography using Protein G HP column. In brief, the protein G column (1 ml) was washed thoroughly with double distilled water (5 column volumes) and equilibrated with 5 column volumes of equilibration buffer (pH 7.0). The periplasmic extract was allowed to pass through the column using a syringe at a sped of 2 ml/min. The column was washed thoroughly by 10 volume of equilibration buffer and bound chimeric Fab was eluted out by 5 volume of elution buffer (pH 2.7). Eluted fractions were immediately neutralized using neutralization buffer (77 pH 9). The eluted fractions were checked on SDA-PAGE.

Assays of the Recombinant Chimeric Fab:

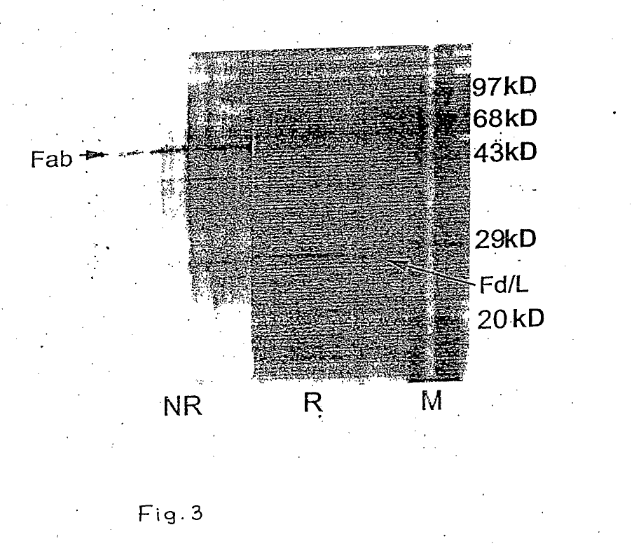

The purified chimeric Fab was resolved by 12% SDS-PAGE, separately in reducing and non-reducing conditions. The resolved protein was stained by silver staining. As shown in FIG. 3, the chimeric Fab is expressed as a heterodimer (˜50 kD) of the chimeric Fd and light chain. In reducing conditions, both the chains were detected in monomeric form (˜25 kD).

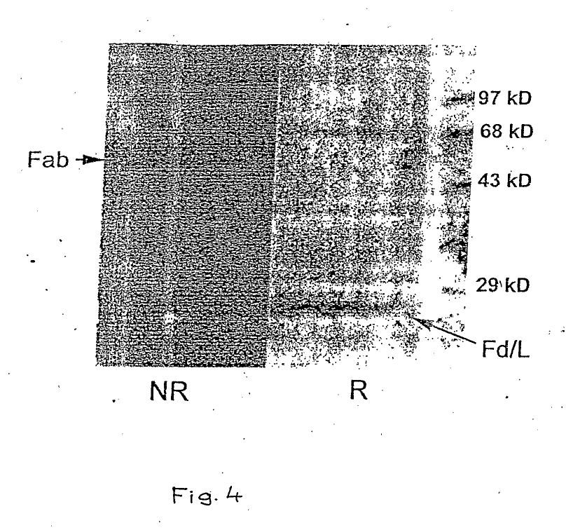

Expression of the heterodimeric chimeric Fab was further confirmed by Western blot in non-reducing conditions (FIG. 4, lane NR). In reducing conditions a band corresponding to monomeric Fd and/light chain was detected (FIG. 4, lane R).

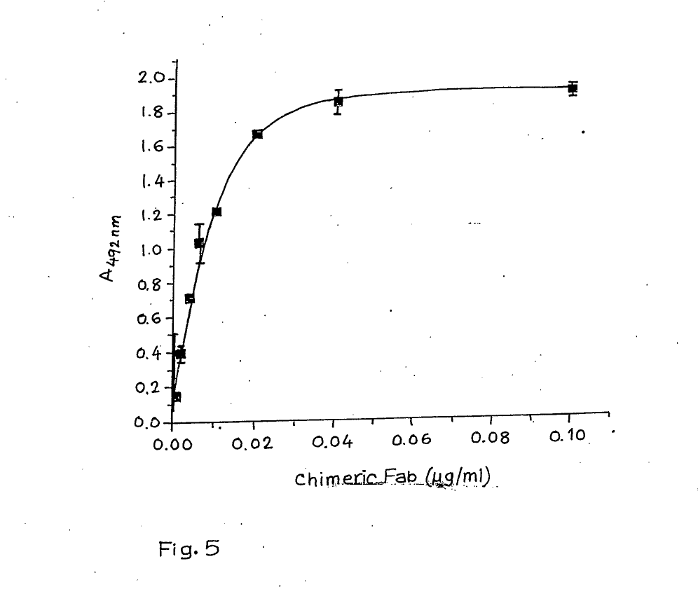

Binding of the chimeric Fab was detected by solid phase ELISA and the result is shown in FIG. 5. As shown in FIG. 5, the binding of the antibody increases with increasing amount of the chimeric Fab, reaching a saturation level as expected for antigen-antibody interactions.

Nucleotide Sequence of the Chimeric Fab:

Antibody Fab fragment is composed of two polypeptide chains: Fd and L.

Nucleotide sequence of the anti-HBsAg mouse-human chimeric Fd chains:

```
    1 gtccagcttc tcgagcccgg ggctgagctg gcgacgcctg gggcctcatt gaagatgtcc
  61 tgcaaggctt ctggctactc atttagcacc tacaacattc actgggtaaa gcagacacct
121 ggacggggcc tggaatggat tggaactatt tatccaggaa ttggtgatac ctcctacaat
181 cagaagttca aaggcaaggc cacattgact gcagacaaat cctccagcac agcctatttg
241 cacctcaaca gcctgacatc tgaggactct gcggtctatt actgtgcaag aagtgacatc
301 tactatggta actacaatgc tttggactac tggggtcaag gaacctcagt cactgtctct
361 tcagcctcca ccaagggccc atcggtcttc cccctggcac cctcctccaa gagcacctct
421 gggggcacag cggccctggg ctgcctggtc aaggactact tccccgaacc ggtgacggtg
481 tcgtggaact caggcgccct gaccagcggc gtgcacacct tcccggctgt cctacagtcc
541 tcaggactct actccctcag cagcgtggtg accgtgccct ccagcagctt gggcacccag
601 acctacatct gcaacgtgaa tcacaagccc agcaacacca aggtggacaa gaaagttgag
661 cccaaatctt gtgacaaaac tagtacatgc
```

Nucleotide Sequence of the Anti-HBsAg Chimeric Light Chain:

```
    1 agatgtgagc tcgtgatgac ccagactcca ctctccctgc ctgtcagtct tggagatcaa
  61 gcctccatct cttgcagagc tagtcagagc attgtccaca gttatggaga cacctatttg
121 gaatggcacc tgcagaaacc aggccagtct ccaaagctcc tgatctacaa agtttccaac
181 cgattttctg gggtcccaga caggttcagt ggcagtggat cagggacaga atttacactc
241 aagatcagca gagtggaggc tgaggatctg ggagtttatt tctgctttca acgttcatat
301 gttccgtgga cgttcggtgg aggcaccaag ctggaactca aacggactgt ggctgcacca
361 tctgtcttca tcttcccgcc atctgatgag cagttgaaat ctggaactgc ctctgttgtg
421 tgcctgctga ataacttcta tcccagagag gccaaagtac agtggaaggt ggataacgcc
481 ctccaatcgg gtaactccca ggagagtgtc acagagcagg acagcaagga cagcacctac
541 agcctcagca gcaccctgac gctgagcaaa gcagactacg agaaacacaa agtctacgcc
601 tgcgaagtca cccatcaggg cctgagttcg cccgtcacaa agagcttcaa caggggagag
661 tgt
```

Amino Acid Sequence of the Chimeric Fab:

Antibody Fab fragment is composed of two polypeptide chains: Fd and L. Amino acid sequence of the anti-HBsAg mouse-human chimeric Fd chains:

VQLLEPGAELATPGASLKMSCKASGYSFSTYNIHWVKQTPGRGLEWIGTI
YPGIGDTSYNQKFKGKATLTADKSSSTAYLHLNSLTSEDSAVYYCARSDL
YYGNYNALDYWGQGTSVTVSSASTKGPSVEPLAPSSKSTSGGTAALGCLv
KDYFPEPVTVSWNSGALTSGVHTFPAVLQSSGLYSLSSVVTVPSSSLGTQ
TYICNVNITKPSNTKVDKKVEPKSCDKTSTC

Amino Sequence of the Anti-HBsAg Chimeric Light Chain:

RCELVMTQTPLSLPVSLGDQASJSCRASQSIVHSYGDTYLEWHLQKPGQS
PKLLIYKVSNRFSGVPDRFSGSGSGTEFTLKISRVEAEDLGVYFCFQRSY
VPWTFGGGTKLELKRTVAAPSVFIFPPSDEQLKSGTASVVCLLNNFYPRE
AKVQWKVDNALQSGNSQESVTEQDSKDSTYSLSSTLTLSKADYEKHKVYA
CEVTHQGLSSPVTKSFNRGEC

The Fab is formed by the precise covalent linkage of the two polypeptide fragments (chemeric Fd and chemeric light chain) at the carboxy terminal of both the fragments. This is via the SH group on the said chain of the amino acid cysteine present in each of the chains at the carboxy end. These two SH groups are condensed from a disulphide (S—S) bond in the proper redox environment to make a functional Fab.

TABLE 1
Oligonucleotide primers used for generation
of the chimeric Fd and light chain

| PRIMER | SEQUENCE |
|---|---|
| 5′ primer for V_{H} | 5′-AG GTC CAG CTT CTC GAG |
| (5H23M) | CCC GGG GC-3′ |
| 3′ primer for V_{H} (Fd3) | 5′-CGA TGG GCC CTT GGT GGA |
| | GGC TGA AGA GAC AGT GAC |
| | TGA GGT TCC-3′ |
| 5′ primer for C_{H}1 of | 5′-GCA ACC TCA GTC ACT GTC |
| human IgG1 (Fd5) | TCT TCA GCC TCC ACC AAG GGC |
| | CCA TCG-3′ |
| 3′ primer for C_{H}1 of | 5′-GCA TGT ACT AGT TTT GTC |
| of human IgG1 (CG1Z) | ACA AGA TTT GGG-3′ |
| 5′ primer for V_{L} | 5′-CCA GAT GTG AGC TCG TGA |
| (5L35) | TGA CCC AGA CTC CA-3′ |
| 3′ primer for V_{L} (K3) | 5′-CAG ATG GTG CAG CCA CAG |
| | TCC GTT TGA GTT CCA GCT |
| | TGG-3′ |
| 5′ primer for C_{L} of | 5′-CCA AGC TGG AAC TCA AAC |
| human K chain (K5) | GGA CTG TGG CTG CAC CAT |
| | CTG-3′ |
| 3′ primer for C_{L} of | 5′-GCG CCG TCT AGA ATT AAC |
| human K chain (Ck1d) | ACT CTC CCC TGT TGA AGC TCT |
| | TTG TGA CGG GCG AAC TCA G-3′ |

The Fab or fragment antigen binding is a combination of the light and the first two domains of heavy chains in a very precise manner. Individually these light and heavy chains will fold in a manner that is very different from the manner they will fold in combination with each other and thus have no desired biological properties. The light chain and heavy chain have to be linked covalently in a manner that will be supportive of their active and folded nature and stabilize the same. This is through the SH groups present on the amino acid cysteine near the carboxy terminal end of the both the molecules. There are numerous cysteins in each of the chains, most form intra chain disulphide bond. However there is only one interchain disulphide bond. This can be formed only the precise alignment of the two relevant cysteines or else the molecule will be mis-folded and have no functions and also itself be antigenic.

In order to make the Fab molecule, the inventors here have adopted a recombinant approach, where individual fragments have been cloned in a vectors specially designed for the purpose. Those fragments have been expressed in a host bacterium (E. coli). This particular strain of E. coli has a periplasmic space which provides an environment of a particularly favourable redox potential that will facilitate the proper interaction of the fragments, their functionally active alignment and the formation of an inter-chain disulplude bond to make a complete and functional Fab fragement.

It is to be noted that the formulation of the present invention is susceptible to modifications, adaptations and changes by those skilled in the art. Such variant formulations are intended to be within the scope of the present invention which is further set forth under the following claims:

We claim:

1. A recombinant chimeric Fab antibody comprising a recombinant Fd and a recombinant chimeric light chain which binds to hepatitis B surface antigen with high affinity, generated by fusing variable region genes (V_{H} and V_{L}) of an anti-HBsAg mouse antibody 5S and constant region genes of human, CH1 region of human IgGI and the CL region of human kappa chain, wherein mouse V_{L} and human CL are linked by overlap PCR to generate chimeric light chain and mouse V_{H} and CH₁ are linked by overlap PCR to generate chimeric Fd.

2. A recombinant chimeric Fab antibody as claimed claim 1, wherein individual fragments have been cloned in a specially designed vector and expressed in a host bacterium, a particular strain of E. Coli having a periplasmic space, which provides an environment of a particularly favorable redox potential, wherein that redox potential facilitate the proper interaction of fragments, their functionally active alignment and the formation of an interchain disulphide bond to make a complete and functional Fab fragment.

3. A recombinant chimeric Fab antibody as claimed in claim 1, wherein only the Fab can bind to Hepatitis B surface antigen and not the individual light and heavy chains.

4. A recombinant chimeric Fab antibody as claimed in claim 1, wherein it has been cloned and expressed in such a fashion that it can be further manipulated to generate a complete chimeric antibody by fusing the chimeric Fd with human Fc fragment.

5. A recombinant chimeric Fab antibody as claimed in claim 1, wherein it has unique mouse variable regions fused to human constant domains, which can be the starting Material for generation of a therapeutically functional full-length recombinant antibody against Hepatitis B surface antigen or for developing any other kind of therapeutic molecule, which could function with or without modification, conjugations etc.

6. A recombinant chimeric Fab antibody as claimed in claim 1, wherein because of its chimeric nature it is less immunogenic than a mouse antibody and as generated by recombinant means is safer and cheaper than currently used human polyclonal antibody.

7. A recombinant chimeric Fab antibody wherein it can be used either on its own or as a starting material for another molecule, with or without appropriate modifications or conjugations for developing a diagnostic reagent for the detection of Hepatitis B surface antigen.
